(19) United States
(12) Patent Application Publication
(10) Pub. No.: US 2026/0286286 A1
(43) Pub. Date: Sep. 24, 2026

(54) Method to Form Microbial Biofilms on Functionalized Hydrogel Polymer Particles for Diagnostics and Cell Culture Applications

(71) Applicant: CERES NANOSCIENCES, INC., MANASSAS, VA (US)

(72) Inventors: Anurag Patnaik, Manassas, VA (US); Ben Lepene, Manassas, VA (US); Patrick Andersen, Manassas, VA (US); Saswata Kumar Sahoo, Manassas, VA (US)

(21) Appl. No.: 19/571,079

(22) Filed: Mar. 18, 2026

Related U.S. Application Data
(60) Provisional application No. 63/773,819, filed on Mar. 18, 2025.

(51) Int. Cl. C12N 1/20; C12N 1/16; C12N 5/09; C12Q 1/18; G01N 33/543

(52) U.S. Cl. CPC C12N 1/20; C12N 1/16; C12N 5/0693; C12Q 1/18; G01N 33/54326

(57) ABSTRACT

Described herein are methods, compositions and systems for the capture, enrichment, culture, and manipulation of microbial cells and biofilms, cancer cells, and extracellular vesicles from liquid sample matrices. The disclosed methods employ magnetically responsive hydrogel particles comprising cationic, anionic, or zwitterionic polymeric materials conjugated to magnetic cores and functionalized with chemical moieties that promote electrostatic and physicochemical interactions with target cell membranes. These interactions enable selective attraction and capture of viable cells and associated cellular components based on membrane electrochemical properties. Following capture, the particles and associated targets are magnetically separated to concentrate the cells and may be directly subjected to culture conditions to support proliferation and biofilm formation in the presence of the particles. Subsequent manipulation and analysis are performed using magnetic separation steps, without reliance on centrifugation or filtration.

### CONTINUITY

This application claims priority to U.S. Provisional Patent Application titled “A Method to Form Microbial Biofilms on Functionalized Hydrogel Polymer Particles for Diagnostics and Cell Culture Applications,” referenced as U.S. Patent Application No. 63/773,819, filed on Mar. 18, 2025, the contents of which are incorporated herein by reference in their entirety.

### FIELD OF THE PRESENT INVENTION

The present invention relates to microbial capture and magnetic concentration from liquid samples using hydrogel-based magnetic particles, followed by subculturing in the presence of the particles to enable identification and characterization of microbial biomass. More specifically, the invention relates to the use of magnetic polymer particle-based workflows that enable the growth of microbes in association with the particles and promote the generation of biofilms for the characterization and manipulation of the microbes and the biofilm.

### BACKGROUND OF THE PRESENT INVENTION

A microorganism, or microbe, is an organism that cannot be easily seen by the unassisted human eye and may exist as a single cell or as a colony of cells. A microbial cell may also reside in close association with other microbial cells of the same or different species, forming a community on the surface of other materials. Such closely associated microorganisms often embed themselves in an extracellular matrix composed of extracellular polymeric substances (EPSs), which are typically a polymeric combination of extracellular polysaccharides, proteins, lipids, and DNA. Such a community of microorganisms is termed as biofilm. The biofilm enables sharing of nutrients among its resident microorganisms and serves as a physical shield against harmful environmental impacts such as desiccation, antimicrobial agents, infecting viruses, other predatory microorganisms, and attacks from a host body's immune system.

These microbial ecosystems or biofilms are ubiquitous in natural, industrial, and clinical settings and play essential roles in nutrient cycling, wastewater treatment, and bioremediation.

In clinical contexts, biofilms are associated with persistent infections, antimicrobial resistance, and complications in medical devices, making them a significant concern in healthcare.

Their inherent resilience, adaptability, and complex interactions have made biofilms a focal point of extensive research efforts.

Preparing biofilms for study currently involves isolating and stabilizing the microorganisms while preserving their natural structure and interactions within the EPS matrix. Current methods for culture and preparation of biofilm often rely on techniques such as centrifugation, filtration, or selective adhesion to surfaces, such as plastic microplates, glass bioreactors or polysaccharide or polyethylene glycol (PEG) based surfaces. The processes involved in these approaches are often exclusively manual and cannot be automated. For characterization and analysis of biofilms, post-culture of biofilms often involves washing off the free cells floating in the culture media and loosely associated with the biofilm, but not a part of the biofilm. The process requires careful separation of the liquid media from the biofilm residing on a surface. This process is almost manual and requires a large volume of liquid, which poses problems for preventing contamination of the growing biofilm. This manual process also requires careful, watchful handling to prevent biofilm components from being washed off, leading to loss of valuable sample material and reducing the fidelity of observations. There can also be chemical treatments for downstream analysis of biofilm materials. Due to limitations in affordability, scalability, or technique, the processes involved in biofilm analysis frequently result in sample loss or contamination, limiting the effectiveness of those analyses. Various culture-based methods, while foundational, are biased toward cultivable species and do not capture the full diversity present in biofilms. Additionally, microfluidics, used to model biofilm growth and behavior under controlled conditions, often struggles to replicate the complexity and heterogeneity of biofilm environments presented in the natural world. These limitations hinder a comprehensive understanding of biofilm dynamics and interactions and present challenges for their characterization and manipulation.

Accordingly, there remains a need for improved approaches to capture microorganisms and promote biofilm formation on surfaces, enabling biofilms to be processed for characterization, analysis, and manipulation, and enabling standardized, simplified workflows without careful handiwork while preserving the integrity of the formed biofilms. In particular, there is a need for methods that facilitate rapid, reproducible and scalable biofilm culture and processing techniques, using approaches compatible with magnetic handling, automation, and scalable processing, targeted for molecular analysis and manipulation.

Microorganisms may exist as individual planktonic cells in suspension or as surface-associated communities embedded in extracellular polymeric substances (EPS), commonly referred to as biofilms. Such microbial communities are encountered across environmental, industrial, and clinical settings, where they are relevant to nutrient cycling, bioremediation, industrial fouling, persistent infections, and antimicrobial tolerance. Because biofilms exhibit distinct structural, metabolic, and resistance properties compared to their planktonic counterparts, their formation, composition, and behavior are subjects of significant research and practical interest.

Conventional approaches for recovering microorganisms from liquid samples and preparing them for study frequently involve multistep workflows including centrifugation, filtration, sedimentation, or adhesion to fixed substrates such as glass, plastic, membranes, or reactor surfaces. These steps are often followed by manual washing, transfer to a culture system, and subsequent analysis. Such workflows may be labor-intensive, difficult to standardize or automate, and prone to contamination or loss of fragile or loosely associated biomass during handling. Where biofilm formation is the objective, the culture substrate is typically a stationary surface that does not readily permit repeated separation of the growing biomass from surrounding liquid without risk of structural disruption.

Various reported approaches have sought to employ particulate supports, including magnetically responsive particles, in workflows for microbial separation, enrichment, or culture-related applications. Certain known methods describe using charged or functionalized particles to bind microorganisms from liquid media and permit magnetic recovery, including approaches that reduce or eliminate reliance on centrifugation or filtration. Other reported methods describe bead-based formats for supporting microbial attachment or evaluating biofilm-related behavior in assay or screening contexts.

Separately, polymeric and hydrogel-based materials have been described for associating with biological targets, including cells and microorganisms, in aqueous environments. Hydrogel scaffolds and encapsulation systems have also been reported for creating three-dimensional microenvironments in which microorganisms can be confined, maintained, and studied under conditions intended to resemble aspects of natural biofilm habitats. Such systems demonstrate that hydrated, porous matrices can support microbial viability and community development, although they have generally been directed toward in vitro modeling or entrapment rather than toward integrated workflows for recovering microorganisms directly from raw liquid samples.

Although each of these various approaches has contributed to the development of microbial processing technologies, they have generally addressed capture, enrichment, culture, and biofilm development as separate operations performed on different substrates or in different vessels. In many reported workflows, the initial recovery step and the subsequent culture or biofilm formation step are not performed on the same material, requiring intermediate transfer, additional handling, and increased risk of contamination or biomass loss. Available magnetic particulate systems, while effective for initial separation or concentration, may not be configured to also serve as a sustained scaffold for biofilm growth or to maintain structural and functional integrity of the resulting biomass through repeated magnetic wash, which consists of separation of suspended materials under magnetism and withdrawal of the suspending liquid, resuspension in a liquid medium, and transfer cycles. Similarly, existing hydrogel-based biofilm culture scaffolds, while capable of supporting microbial attachment and three-dimensional community development, are generally not designed for direct capture and magnetic concentration of microorganisms from raw or minimally processed liquid samples.

Accordingly, there remains a need for improved compositions and methods that provide a single magnetically addressable substrate capable of functioning both as an initial capture medium for recovering microorganisms from a liquid sample and as a sustained scaffold for subsequent culture and biofilm development on the same material. There remains a further need for such a substrate to preserve the viability and structural integrity of captured and cultured biomass throughout the workflow, and to be compatible with scalable, reproducible, and optionally automated processing, including repeated magnetic handling and downstream molecular, microbiological, or biochemical analysis.

Starting point of almost every processing, analysis or isolation of components of biofilm is the enrichment of the biofilm, which can be termed as “biofilm harvest’. The harvest of biofilms from captured microorganisms are comprised of two steps—(a) capture of microorganisms and subsequent (b) multiplication of microorganisms under suitable conditions conducive for biofilm formation. The standard method for capturing viable microorganisms from any liquid sample involves centrifugation of the liquid sample and precipitation of the microbes or filtering the sample through a membrane of appropriate porosity and thus separating the microbes from the bulk liquid. As mentioned earlier, the biofilm is usually grown by culturing microbes in a bioreactor which has plastic, glass or polymeric surface for attachment of the microbes and the biofilm. By subsequently connecting the capturing of microorganisms, by centrifugation or filtration, and then culturing the captured microbes on an appropriate surface, the biofilm harvest is achieved according to conventional approaches. In summary it can be assessed that at present there is no simple method for an efficient, fast, inexpensive enrichment of bacterial cells and biofilm which get along without a centrifugation or filtration step, and which at the same time can be used for a broad variety of microbes. Therefore, it is the task of the present invention to provide an approach for the unspecific harvest of microbial cells and associated biofilms that get along without a centrifugation or filtration step and thus provide the scopes for automation and scalability.

The concentration and separation of microbes from a liquid by centrifugation relies on the higher forces exerted on the masses of the microbial cells, which range from 10 nm to 750 μm in size, compared to small molecules, proteins, lipids, etc., which are <5 nm in size, during the process. The higher centrifugal forces make the microbial cells precipitate preferentially than inorganic and organic molecules and their aggregates. The microbial precipitate can be isolated from the bulk liquid after the centrifugation process for purification and further downstream processes. The filtration processes rely on selective permeability of microbes based on the sizes through a membrane of specific porosity and thus concentrating the target microbes on the membrane or in the filtrate.

The present invention exploits the chemical and electrical properties of microbial surfaces and EPS components. Microbes can be grouped into viruses, bacteria, archaea, fungi (such as yeasts and molds) and protozoa. The cell surfaces of most viruses, bacteria, yeasts, molds and protozoa carry a net negative electrostatic charge at physiologically relevant or ambient aqueous conditions. Among the anionic membrane components of bacteria, lipopolysaccharides (LPS) of Gram-negative bacteria and lipoteichoic acids (LTA) of Gram-positive bacteria deserve particular importance. If present in blood, LPS and LTA can trigger unregulated immune response in animals known as sepsis, which is a life-threatening condition without any known cure. As such LPS and LTA are considered as pyrogens, which are fever-inducing contaminants, that cause fever, chills, hypotension, and shock when introduced into the body. On the other hand, the EPS matrices of biofilm often possess an overall negative charge. The key polysaccharides in the matrix include alginate, xanthan, cellulose, PeI, PsI, Poly-β-(1→6)-N-acetylglucosamine (PNAG), colonic acid and Levan. Among these polysaccharides, levan and PsI are predominantly neutral, PNAG and PeI are cationic, while the others are predominantly negative in electrostatic character. Moreover, nucleic acid molecules such as DNA (deoxyribonucleic acids) and RNA (ribonucleic acids), also possess net negative charge on the molecules. Thus, polymeric materials possessing net electropositive charge, due to presence of substituted or non-substituted amine groups (—NH₂, HN—R—, —R¹—N—R²—) or imine groups (>C═NH, >C—N═R—) in their architectures, are poised to attract and bind to the microbial surfaces, EPS matrix and anionic microbial cell components such as but not limited to LPS and LTA. Here “R” represent hydrogen alkyl, alkenyl, alkynyl or aromatic group. On the other hand, polymeric materials, containing anionic moieties, such as but not limited to carboxyl groups (—COOH), sulfonic group (—SO₃H), can bind to the electropositive moieties present on microbial surfaces, inside EPS matrices and in the architectures of microbial cell components.

Cancer cells also possess net negative surface charges, which are stronger than that of normal, healthy cells. This negative charge is primarily driven by high glycolysis rates and increased secretion of lactic acid. Moreover, extracellular vesicles (EVs), including exosomes and microvesicles, possess a net negative surface charge under physiological conditions. This negative charge is primarily due to the presence of phospholipids, particularly phosphatidylserine, and sialylated glycoproteins on the lipid bilayer membrane. Thus, polymeric materials possessing net electropositive charge, due to presence of substituted or non-substituted amine groups (—NH₂, HN—R—, —R¹—N—R²—) or imine groups (>C═NH, >C═N—R—) in their architectures, are poised to attract and bind to the membrane surfaces of cancer cells and EVs. Here “R” represents hydrogen alkyl, alkenyl, alkynyl or aromatic group. On the other hand, polymeric materials, containing anionic moieties, such as but not limited to carboxyl groups (—COOH), sulfonic group (—SO₃H), can bind to the electropositive moieties present on the membrane surfaces of cancer cells and EVs.

Magnetic hydrogel particles are specialized hydrogel microspheres designed to rapidly capture, concentrate, and preserve low-abundance analytes from liquid samples which include but not limited to sewage, run off, water from ponds, lakes, wetlands, rivers and sea, food wash, beverages, aqueous liquids used in biological manufacturing or clinical fluids and samples including but not limited to urine, blood, cerebrospinal fluid, sputum, nasal and skin secretions. Based on the specific application, the hydrogel architectures of magnetic hydrogel particles contain chemical baits that attract specific targets which include but are not limited to proteins, lipids, nucleic acids, carbohydrates, metabolic intermediates, microbes, extracellular vesicles and animal cells.

The specialized hydrogel materials of magnetic hydrogel particles can be grown on magnetic particles to produce magnetic hydrogel particles which can be functionalized and utilized in similar manner as described above. The magnetic hydrogel particles have the additional benefit of being responsive under magnetism, which enables the particles to get separated from the bulk liquid with the targeted analytes bound to the particles without requiring centrifugation or filtration process. The magnetic hydrogel particles allow scalability and automation of sample processing.

The inclusion of substituted or non-substituted amine groups (—NH₂, HN—R—, —R¹—N—R²) or imine groups (>C═NH, >C═N—R) in the architectures of hydrogel material of magnetic hydrogel particles, can enable the particles to attract and bind to the microbial surfaces, EPS matrix, cancer cells, EVs and anionic microbial cell components such as but not limited to LPS and LTA. Here “R” represents hydrogen alkyl, alkenyl, alkynyl or aromatic group. On the other hand, polymeric materials, containing anionic moieties, such as but not limited to carboxyl groups (—COOH), sulfonic group (—SO₃H), can bind to the electropositive moieties present on microbial surfaces, inside EPS matrices and in the architectures of microbial or cancer cell components and the components of EVs.

### SUMMARY OF THE INVENTION

The present invention presents novel approach involving magnetic hydrogel particles, which have been developed to enable efficient capture and stabilization of a broad spectrum of microorganisms, cells and EVs directly from liquid samples. These particles are designed to eliminate the necessity for centrifugation or filtration for capturing microbes, animal or plant cells and EVs from a liquid sample. Instead, this novel approach integrates high surface-area porosity with customizable affinity baits, exploiting a combination of chemical interactions (e.g., charge-based or ligand-specific binding) and physical mechanisms (e.g., size-exclusion) to achieve robust, multi-target capture in a single step.

These particles are designed to minimize the need for disruptive sample preparation techniques, preserving the structural integrity of biofilms and maintaining microbial and cellular viability. By providing a versatile surface for microbial and cellular attachment, the particles facilitate controlled biofilm formation, enabling real-time observation and analysis of biofilm characteristics and dynamics.

Leveraging their magnetic properties, these particles streamline the isolation process, overcoming common issues such as sample loss, scalability, lack of scope for automation and possibility of contamination.

In one aspect, this innovation offers a robust, scalable solution to the challenges of biofilm preparation and study, enhancing the efficiency and accuracy of biofilm research and microbial characterization.

In another aspect, this innovation offers a robust, scalable solution to the challenges of cell culture preparation and study, enhancing the efficiency and accuracy of cellular research and characterization of cellular organizations.

In one aspect, this invention provides a unified approach suitable for processing macro-volumes of liquid samples and concentrating populations of microbes and animal or plant cells, such as but not limited to cancer cells, for their culture in a lower volume and facilitating the formation of biofilms and cellular organizations.

In one aspect, the invention provides an approach for processing a liquid sample to capture the resident cellular targets, including bacteria, fungi, archaea, viruses, protozoa, animal cells or their cellular components. The method comprises: (a) contacting the liquid sample with magnetically responsive hydrogel particles; (b) capturing the cellular targets using those magnetically responsive hydrogel particles; (c) magnetically separating particle-bound cellular targets from the liquid sample partially or completely to concentrate and or isolate the microbial targets from the liquid sample; (d) detaching the cells from the particles or not detaching the cells from the particles; (e) placing the cells associated or non-associated with the particles in a growth promoting media or preservation media or processing the cells associated or non-associated with the particles for detection, identification and characterization of the cellular biomass; and/or (f) letting the cells multiply in association or non-association with the particles; (g) then subsequent analysis and/or manipulation of the cells and biomass by either separating the cells and cellular biomass from the particles or not separating the cells and the biomass from the particles.

In another aspect, the invention provides magnetically responsive hydrogel particles comprising a polymeric hydrogel matrix that is intrinsically magnetic or magnetically addressable via association with a magnetic component. In certain embodiments, the hydrogel comprises one or more structural monomers and one or more affinity moieties selected to promote binding to cellular surface components or components of EPS matrix in liquid samples, thereby enabling rapid magnetic separation of captured microbial targets from bulk fluid. In certain embodiments the association between the polymeric hydrogel matrix and the magnetic component is a covalent chemical bond.

In certain embodiments, the approach enables concentration of microbial and cellular targets from sample volumes of about 0.1 mL to greater than 100 mL into culture volumes of about 500 μL to greater than 100 mL. In these embodiments, microbial or animal or plant cells remain viable during capture and washing enabling subsequent generation of progenies, biofilm and cellular organizations from the captured cells in the growth media in presence of the particles.

In certain embodiments, the cellular organizations formed in association with the magnetic particles can be separated and isolated from the bulk liquid using magnetic forces. In certain embodiments, the magnetically separated and isolated cellular organizations remaining in association with the magnetic particles can be resuspended in another liquid after introduction of that new liquid and withdrawal of the magnetic forces and then separated and isolated again under magnetic forces—thus enabling washing of the cellular organizations, such as but not limited to biofilms, repeatedly. In certain embodiments the cellular organizations in presence of the magnetic particles can be studied using physical and chemical analytical techniques or can be lysed via thermal, chemical, mechanical, or enzymatic disruption to render biochemical components, such as but not limited to nucleic acids, for analysis. In certain embodiments, the cells and the cellular organizations can be separated from the magnetic particles prior to studying them using physical and chemical analytical techniques or can be lysed via thermal, chemical, mechanical, or enzymatic disruption to render biochemical components, such as but not limited to nucleic acids, for analysis.

In certain embodiments the magnetic particles enable concentration of microbial and cellular targets from liquid samples. In certain embodiments the liquid sample can be but is not limited to biological, environmental, industrial, and clinical matrices, including whole blood, urine, serum, plasma, cerebrospinal fluid, saliva, bronchoalveolar lavage fluid, sputum, viral transport media, wastewater, surface water, potable water, culture media, and food matrices.

The following brief and detailed descriptions of the drawings are provided to explain possible embodiments of the present invention but are not provided to limit the scope of the present invention as expressed herein this summary section.

### BRIEF DESCRIPTION OF DRAWINGS OF THE PRESENT INVENTION

The accompanying drawings, which are incorporated herein and form a part of the specification, illustrate the present invention and, together with the description, further serve to explain the principles of the invention and to enable a person skilled in the pertinent art to make and use the invention.

The present invention will be better understood with reference to the appended drawing sheets, wherein:

FIG. 1 shows two magnetic devices constructed for separation of magnetic particles under magnetic force inside a microplate. (A) was constructed for a 6 well microplate and (B) was constructed for a 12 well microplate.

FIG. 2 displays the results obtained from bacterial (Klebsiella p. 13884) survival assay using resazurin metabolism as indicator of survival immediately after capture by magnetic hydrogel particles. The correlations depict how the time (threshold time) required to reach a specific fluorescence, due to resazurin reduction by living cells, is correlated to the initial population (CFU) of microbes in presence of (A) NT-B, (B) NT-C and (C) in absence of particles. These correlations were used to estimate the bacterial population in the samples, and the results were shown in the plot (D).

FIG. 3 shows biofilm produced by green fluorescence protein (GFP) producing E. coli. (A) shows the microplate after washing off the planktonic cells and illuminating the plate with near UV blue light. Wells 3A and 3B display glowing green fluorescent biofilm submerged in aqueous medium. The fluorescence was produced from GFP present inside the bacterial cells residing in the biofilm. The fluorescence cannot be observed adequately in the wells of 1A, 1B and 2A, 2B which were also supposed to contain biofilm with residing bacteria but the magnetic hydrogel particles attenuate fluorescence. Samples of row C do not contain bacteria, while column 4 contains sterile water. (bottom panel) shows correlations between bacterial concentration and fluorescence intensities in presence of (B) NT-B, (C) NT-C and (D) in absence of particles. The correlations were used to determine population of bacteria in the biofilms, and the results were shown in (E).

FIG. 4 demonstrates the spectral analysis of crystal violet (CV). (A) absorbances were read at different wavelengths 300 nm-800 nm. λₘₐₓ was found to be ~585 nm. (B) shows the correlation between different concentrations of CV and their absorbance values.

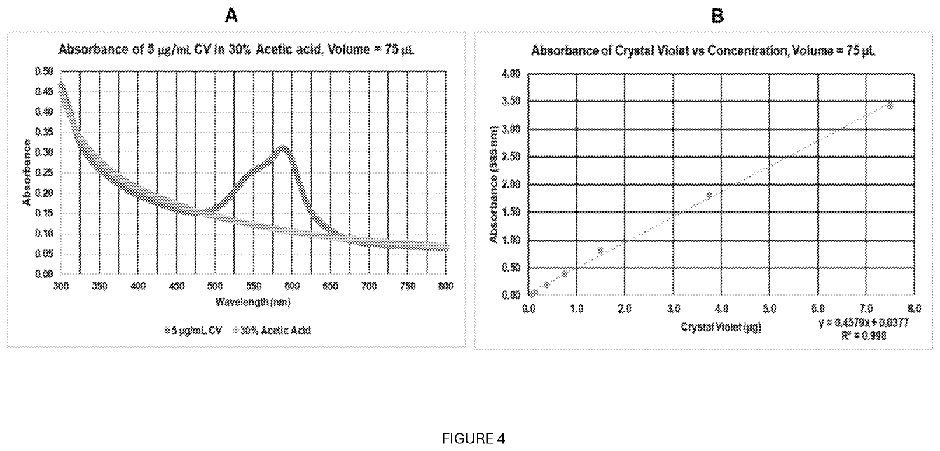

FIG. 5 shows the effect of different growth media on the amounts of biofilms harvested by magnetic hydrogel particles for Klebsiella sp. 13884 cells. NT-C was used in (A), while in (B) both NT-B and NT-C were used.

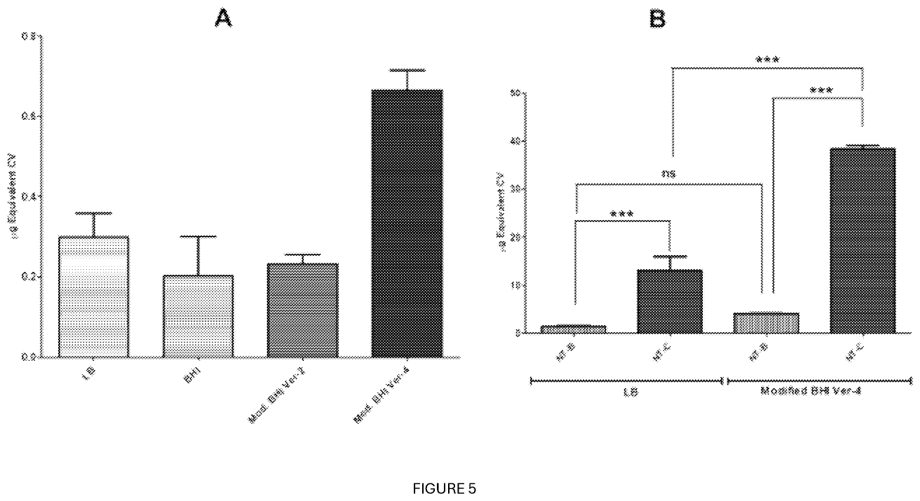

FIG. 6 demonstrates how amounts of biofilm harvested (for Klebsiella p. 13884) depend on the volumes used for specific magnetic hydrogel particles. Increased volumes of particles can enable increased amounts of biofilm harvest.

FIG. 7 shows amounts of biofilm (for Klebsiella p. 13884) harvested by using magnetic hydrogel particles (NT-B and NT-C) vs not using magnetic hydrogel particles.

FIG. 8 shows the results after biofilms (for E. coli 25922) harvested by using or not using magnetic hydrogel particles were treated with different concentrations of an antibiotic—levofloxacin (LVFX). (A) No large variation was observed in the degrees of inhibition for planktonic cells, (B) demonstrates that while the magnetic hydrogel particles preserved biofilms, significant eradication was observed in case of biofilms not attached to particles when treated with 100 μg/mL and 10 μg/mL of LVFX. (bottom panel) The correlations depict how the time (threshold time) required to reach a specific fluorescence, due to resazurin reduction by living cells, is correlated to the initial population (CFU) of microbes in presence of (D) NT-B, (E) NT-C and (F) in absence of particles. These correlations were used to determine surviving bacterial population in the biofilm samples and the results were displayed in (C).

FIG. 9 shows biofilm (for Candida albicans) harvested by using magnetic hydrogel particles vs not using particles.

### DETAILED DESCRIPTION OF THE PRESENT INVENTION

The present specification discloses one or more embodiments that incorporate the features of the invention. The disclosed embodiment(s) merely exemplify the invention. The scope of the invention is not limited to the disclosed embodiment(s).

References in the specification to “one embodiment,” “an embodiment,” “an example embodiment,” etc., indicate that the embodiment described may include a particular feature, structure, or characteristic, but every embodiment may not necessarily include the particular feature, structure or characteristic. Moreover, such phrases are not necessarily referring to the same embodiment, Further, when a particular feature, structure, or characteristic is described in connection with an embodiment, it is submitted that it is within the knowledge of one skilled in the art to affect such feature, structure, or characteristic in connection with other embodiments whether or not explicitly described.

The approach described herein are suitable for culturing cells for identification, diagnostics, research and development applications, including epidemiology, wastewater surveillance, environmental monitoring, food safety testing, and biodefense, and support detection, identification, and isolation of biomolecules for analysis, nucleic acid isolation amplification, sequencing and manipulation.

As used herein, “sample” refers to a biological, environmental, or food matrix suspected of containing one or more microbial or cellular or cell component targets, including, without limitation, blood, plasma, serum, urine, saliva, cerebrospinal fluid, bronchoalveolar lavage fluid, sputum, viral transport medium, wastewater, surface water, groundwater, soil, sludge, industrial or agricultural water, and liquid or semi-liquid food matrices.

“Microbial target” and “microorganism” refer to viruses, bacteria, fungi, yeast, algae, protozoa, or parasites and may include viable organisms, partially intact organisms, fragments, or complexes.

“Cellular target” refers to microbial, plant, or animal cells including but not limited to cancer cells, and may include viable cells, partially intact organisms, fragments, complexes or components.

“Cellular components” refers to proteins, lipids, carbohydrates, nucleic acids, metabolites derived from animal, plant, and microbial cells. In one embodiment, “cellular components” also include membrane bound organizations derived from a cell which may include but not limited to mitochondria, nucleus, Golgi bodies and extracellular vesicles. In another embodiment, “cellular components” also include artificially constructed proteins, lipids, carbohydrates, nucleic acids, metabolites, membrane bound organizations such as but not limited to extracellular vesicles, Golgi bodies and lipid nanoparticles.

“Nucleic acid” refers to DNA or RNA, including single-stranded or double-stranded forms and fragmented or partially degraded forms.

“Biofilm” refers to a community of microorganisms that adhere to living or non-living surfaces and are embedded in within an extracellular polymeric substance (EPS) matrix produced by the microorganisms. A biofilm may also contain animal cells, such as cancer cells, in mutually beneficial relation with the microorganism(s) within the biofilm.

“Cellular organization” refers to the spatial arrangement, interaction, and structural characteristics of animal cells or plant cells, as they grow in vitro, typically forming monolayers or multilayers.

In one aspect, the invention provides an approach for design and synthesis of magnetic particles coated with hydrogel polymer material containing cationic, anionic or zwitterionic moieties. Such particles are referred to as magnetic polymer particles or magnetic hydrogel particles. In certain embodiments the hydrogel polymer material is conjugated with magnetic particles via covalent chemical bond. In certain embodiments, the cationic moieties are without limitation comprised of substituted or non-substituted amine groups (—NH₂, —NH—R—, —R¹(N—)—R²—, (—N+)—(R¹—)(R²—)(R³—) or imine groups (>C═NH, >C═N—R), guanidinium group [(R¹R²N)(R³R⁴N)C═NR⁵], pyridinium group (C₅H₅N⁺R), sulfonium group (—S⁺R¹R²R³), carbenium or oxonium groups or a combination of these groups. In certain embodiments the anionic moieties are without limitation comprised of carboxyl (—COOH) group, sulfonate and sulfate (RSO₃⁻ and ROSO₃⁻ respectively) groups, phosphate (ROPO₃²⁻) group, or alkoxide (RO⁻) group or a combination of two or more of such groups. Here “R” represents hydrogen alkyl, alkenyl, alkynyl, or aromatic group. In certain embodiments, the zwitterionic moiety may, without limitation, comprise of one or multiple of the mentioned cationic groups and one or multiple of the mentioned anionic groups.

In one aspect, the invention provides an approach for introduction of magnetic hydrogel particles to a liquid sample suspected of containing microbial and/or cellular targets and components with the objective of capturing the targets on the particles. In some embodiments, post capturing of cellular targets, the particles can be separated by centrifugation, filtration, precipitation under gravitational force or by application of magnetic force, along with the cellular targets attached with the particles. In certain embodiments the separated particles, attached with cellular targets, can be isolated from the liquid sample for downstream processing.

In one aspect the downstream processing may comprise of degradation of cellular targets by physical or chemical process(es) and release of the biomolecules in appropriate media followed by appropriate biochemical and molecular analysis to detect, identify, characterize or manipulate the biomolecules.

In another aspect, the downstream processing may comprise of detaching the cellular targets from the particles and placing those cells in appropriate media for preservation of the targets. In some embodiments, the cellular targets suspended in the preserving media may be further processed for their identification, characterization and manipulation.

In another aspect, the downstream processing may comprise of detaching the microbial and/or animal cells from the particles and placing those cells in appropriate media for preservation of the cells. In some embodiments, the cells suspended in the preserving media may be further processed for their identification, characterization and manipulation.

In another aspect, the downstream processing may comprise of placing the cellular targets, without separated from the magnetic hydrogel particles, in appropriate media for preservation of the targets. In some embodiments, the cellular targets suspended in the preserving media may be further processed for their identification, characterization and manipulation.

In another aspect, the downstream processing may comprise of placing the microbial and/or animal cells, without separated from the magnetic hydrogel particles, in appropriate media for preservation of the targets. In some embodiments, the cellular targets suspended in the preserving media may be further processed for their identification, characterization and manipulation.

In another aspect, the downstream processing may comprise of detaching the microbial and/or animal cells from the particles and placing those cells in nutritious media for culturing of the cells. In some embodiments, the culturing process may be preceded by a sorting process to separate cells into subpopulations and thus allowing culturing of single strain of cells. In another embodiment, the culturing may be preceded by partial or no sorting of cells and thus allowing culturing of mixed strains of cells. In some embodiments, the culturing of cells may enable production of biofilms or cellular organizations. In some embodiments, the cultured cells, biofilms and/or cellular organizations may be subjected to further downstream processing.

In another aspect, the downstream processing may comprise of placing the microbial and/or animal cells in nutritious media for culturing of the cells. In some embodiments, the culturing process may be preceded by a sorting process to separate cells into subpopulations and thus allowing culturing of single cells. In another embodiment, the culturing may be preceded by partial or no sorting of cells and thus allowing culturing of mixed cells. In some embodiments, the culturing of cells may enable production of biofilms or cellular organizations. In some embodiments the biofilms or the cellular organizations may be attached to the particles. In some embodiments, the cultured cells, biofilms and/or cellular organizations, in association or not in association with the particles, may be subjected to further downstream processing.

The invention will be further described with reference to the following examples; however, it is to be understood that the invention is not limited to such examples.

### Materials and Methods

### Example 1: Materials and Instrumentation

Magnetic iron oxide infused carboxylated polymeric particles were purchased from commercial vendors. The median diameter of the particles may range from 200 nm to 20 μm.

Microbial strains were procured from American Type Culture Collection (ATCC) and cultured in appropriate growth medias. The derived progenies of the specific strains are mentioned simply as the ATCC strain identification—for example subcultures of E. coli from ATCC 25922 is simply mentioned as E. coli 25922, and so forth.

Below is a list of chemicals and materials with their vendors used during the experiments of this invention.

TABLE 1

| Chemical Name | Abbreviation | Vendor |
|---|---|---|
| N-isopropyl acrylamide | NIPAM | TCI America |
| N.N′-methylenebisacrylamide | BIS | MilliporeSigma |
| Acrylic acid | NA | MilliporeSigma |
| Potassium persulfate | KPS | MilliporeSigma |
| Acetic acid | AcA | MilliporeSigma |
| Tween-80 | Tw80 | MilliporeSigma |
| 96 well polypropylene black | back pp | Greiner Bio-One |
| microplates | microplate | |
| 96 well polypropylene clear | clear pp | Greiner Bio-One |
| microplates | microplate | |
| 12 well clear polystyrene microplate | | Greiner Bio-One |
| 6 well clear polystyrene microplate | | Greiner Bio-One |
| Hydrochloric acid | HCl | Carolina Biological |
| | | Supply |
| Sodium hydroxide | NaOH | Thermo Fischer |
| | | Scientific |
| Liquid culture medias | | Teknova |
| Media agar plates | | Teknova |
| Glycerol | | Teknova |
| Luria Bertani broth | LB | Teknova |
| Cation adjusted Mueller Hinton Broth | MHBII | Teknova |
| Brain Heart Infusion Broth | BHI | Teknova |
| Brain Heart Infusion broth powder | BHI powder | MilliporeSigma |
| Tryptic soy broth | TSB | Teknova |
| Neutralizing Broth powder | NB powder | BD Difco |
| Sodium chloride | NaCl | MilliporeSigma |
| Brain Heart Infusion (Porcine) | | MilliporeSigma |
| Fructose | | MilliporeSigma |
| Glucose | | MilliporeSigma |
| Ampicillin | Amp | MilliporeSigma |
| Peptone | | MilliporeSigma |
| Meat Extract | | MilliporeSigma |
| Yeast Extract | | MilliporeSigma |
| Isopropyl β-D-1-thiogalactopyranoside | IPTG | Teknova |
| Crystal Violet | CV | MilliporeSigma |
| Hank's Balanced Salt solution | HBBS | Gibco |
| Levofloxacin | LVFX | MilliporeSigma |

The buffers and media were either procured from vendors as mentioned in Example 1 or being supplemented with mentioned additives or constituted using ingredients, listed in Example 1, as described and then filtered through 0.2 μm PES membrane filter units.

Dilution buffer was prepared by supplementing HBBS with 0.1% (w/v) peptone and 0.1% Tw80, followed by filter sterilization. The ionic formulation and the glucose content of HBBS are similar to physiological conditions. Supplementation with small amounts of peptone provides a nitrogenous nutrition source for the suspended bacteria for several hours at room temperature without invoking significant growth. Supplementation with small amount of Tw80 prevents aggregation of cells in the suspension.

Modified BHI version-1 (Mod. BHI V-1) was prepared by dissolving 18.5 g of BHI powder in water. Then the pH was adjusted to 8.0 with aq. NaOH. The volume was made up to 500 mL with water and then filter sterilized.

2× Neutralizing buffer broth (2×NBB) was prepared by dissolving 5.2 g of NB and 32 g of BHI powder in 500 mL water followed by filter sterilization. NB was supposed to neutralize the bacteriostatic and bactericidal effects of adverse chemicals and support recovery of microbes from surfaces.

Modified BHI version-2 (Mod. BHI V-2) was prepared by dissolving 18.5 g of BHI powder in water. Then the pH was adjusted to 8.5 with aq. NaOH. The volume was made up to 500 mL with water and then filter sterilized. Higher pH was supposed to enhance biofilm production in Klebsiella p.

Modified BHI version-3 (Mod. BHI V-2) was prepared by dissolving brain heart infusion (porcine) powder (8.75 g), Na₂HPO₄ (1.25 g), Fructose (1 g), meat peptone (5 g) and NaCl (2.5 g) in water. Then the pH was adjusted to 8.5 with aq. NaOH. The volume was made up to 500 mL with water and then filter sterilized. Higher pH and presence of fructose was supposed to enhance biofilm production in Klebsiella p.

Modified BHI version-4 (Mod. BHI V-4) was prepared by dissolving brain heart infusion (porcine) powder (8.75 g), Na₂HPO₄ (1.25 g), Fructose (1 g), meat peptone (2.5 g), yeast extract (2.5 g) and NaCl (2.5 g) in water. Then the pH was adjusted to 8.5 with aq. NaOH. The volume was made up to 500 mL with water and then filter sterilized. Higher pH and presence of fructose was supposed to enhance biofilm production in Klebsiella p., and presence of yeast extract was supposed to support growth of yeasts.

Multimode microplate reader—Varioskan Lux operated via software Skanlt RE (7.02) was used for spectral readings. The results were exported to Microsoft Excel and then analyzed. The data was then plotted using Prism 5 (Graphpad Inc.) and statistical analysis were conducted. The plots displays the means of the data with the error bars depict the standard error. Where applicable, one-way ANOVA with Tukey post test was conducted to find any statistically significant difference (confidence interval=95%) within the means and the results were provided as non-significant (ns) for p>0.05, otherwise is significant with * (0.005<p<0.05), ** (0.0001<p<0.005) and *** (p<0.0001).

Magnetic devices were constructed for separating particles in the 12 well plates and 6 well plates by sticking magnets over the lids for respective plates (Figure-1). When a plate is placed on an appropriate magnetic device, the magnetic hydrogel particles stick close to the magnets in the wells of the plate, allowing removal of solvents without particles being flown out and getting lost.

### Spectral Studies of Crystal Violet Dye (CV)

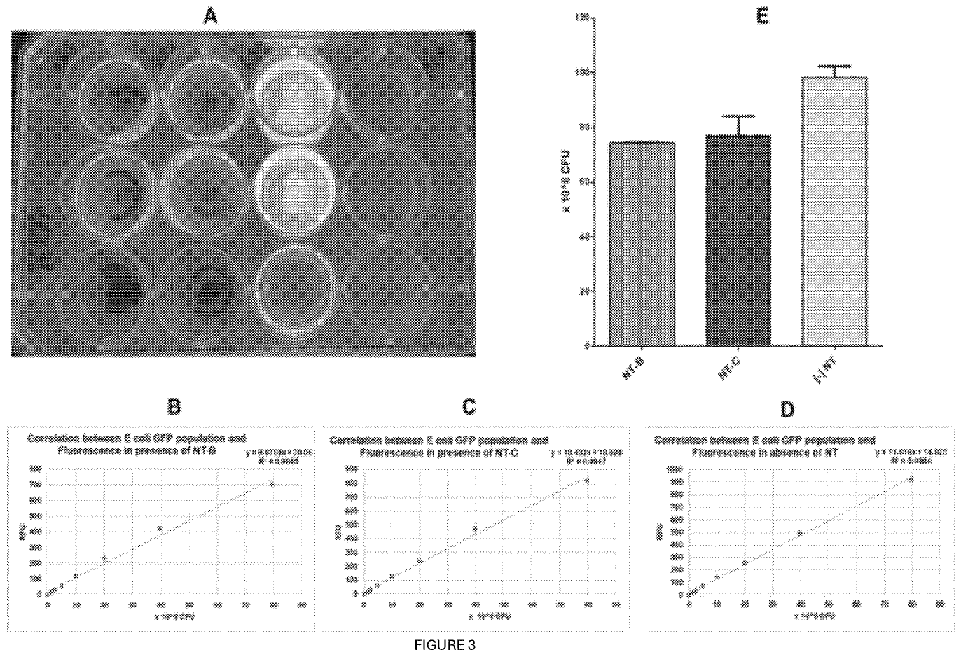

Stock solution of CV was prepared in 30% AcA at 1 mg/mL. Then dilutions of CV were prepared in the same solvent. 75 μL of the samples were pipetted in to separate wells of a 96 well clear pp microplate. At first absorbance was read for blank and 5 μg/mL samples between 300 nm-800 nm to find the wavelength (λₘₐₓ) at which CV absorbs the most. The λₘₐₓ was found to be ~545 nm, which matches with established λₘₐₓ for CV (FIG. 3). Then the absorbances of samples in all the wells were measured at 545 nm. The result shows a linear correlation between the absorbance and the [CV] from 100 μg/mL to 1 μg/mL. The linear correlation between concentration of CV and their absorbances enabled us to quantify biofilm in a sample in terms of “μg equivalence of CV”. Measurement of absorbances of CV-stained biofilm samples in a microplate in the subsequent examples is always accompanied with measurement of absorbances of dilutions of CV (100 μg/mL-1 μg/mL) in the same microplate for generating a standard curve for quantifying the amount of biofilm in the samples.

### EXPERIMENTAL EXAMPLES

The following examples are provided for purposes of illustration and description and are not intended to limit the scope of the present invention.

### Example 1: Synthesis of Magnetic Hydrogel Particles

The carboxy groups of the magnetic iron oxide infused carboxylated polymeric particles (preferably 400 nm-7 μm diameter) were amidated by using excess amounts of allylamine hydrochloride and N-(3-Dimethylaminopropyl)-N′-ethylcarbodiimide hydrochloride (EDC) in an aqueous solution buffered by 4-Morpholineethanesulfonic acid (MES) at pH=5-6. The amidated particles are referred to as allylamide particles, whose polymeric surface had been modified to possess vinyl groups, which act as the point of attachment for growing polymeric materials on the particles' surfaces. These allylamide particles were used as magnetic cores to grow hydrogel polymer materials, and thereby coating the surfaces of the particles to form magnetic hydrogel particles.

In one embodiment, the allylamide particles were introduced to a jacketed glass reactor with excess water and subjected to heating at 50-75° C. along with degassing by bubbling nitrogen. Then a concentrated solution containing NIPAM, BIS and acrylic acid was introduced into the reaction mixture along with a separate solution of KPS. The polymer was allowed to form on the allylamide particles for several hours before cooling down the reaction mixture. The polymerization reaction created co-polymeric NIPAM and acrylic acid hydrogel crosslinked with BIS and covalently bound to the polymeric architecture of the magnetic iron oxide infused polymeric particles. The particles were subsequently washed with diluted sodium hydroxide (aq. NaOH) solution several times using magnetic separation processes and then ultimately suspended in aqueous medium. Small aliquots from the suspension were dried and then weighed to determine the concentration of the hydrogel coated magnetic particles in the suspension. The concentration of the suspension was finally adjusted appropriately. The formed hydrogel layer encapsulating magnetic particles contain anionic carboxyl groups. In the next step, these carboxyl groups were amidated with amine containing polymers in MES buffered aqueous solution facilitated by EDC. Now, these polyamine conjugated particles were washed with diluted hydrochloric acid (aq. HCl) several times before finally being suspended in aqueous media. We refer to this polyamine containing magnetic hydrogel particles to be Magnetic hydrogel-B (NT-B) particles. In most cases batch #7348 was used.

In another embodiment, the allylamide particles were introduced to a jacketed glass reactor with excess water and subjected to heating at 50-75° C. along with degassing by bubbling nitrogen. Then a concentrated solution containing NIPAM, BIS and a monomer, containing a quaternary ammonium group, was introduced into the reaction mixture along with a solution of potassium persulfate (KPS). The polymer was allowed to form on the allylamide particles for several hours before cooling down the reaction mixture. The particles were subsequently washed with aq. HCl solution several times using magnetic separation processes and then ultimately suspended in aqueous medium. We refer to this quaternary ammonium containing magnetic hydrogel particles to be Magnetic hydrogel-C (NT-C) particles. In most cases batch #7517 was used.

The NT particles were washed with concentrated guanidine thiocyanate solution followed by several washes with sterile aqueous solution for sterilization before finally being suspended in sterile aqueous media. Then concentration was adjusted appropriately.

### Example 2: Culture of Microorganisms

Cells from ATCC were first rehydrated with MHBII and then inoculated onto TSB agar plates following streak plate protocol. The rehydrated cells were added with glycerol up to a final concentration of ~20% (v/v), aliquoted into small microcentrifuge tubes and stored at −80° C. These aliquots are designated as generation-0 (G-0).

Several colonies from the overnight grown streak plates were selected and resuspended in MHBII supplemented with 0.1% Tw80 (MHBII+Tw80). The suspension was supplemented with glycerol up to a final concentration of ~20% (v/v), aliquoted into small microcentrifuge tubes and stored at −80° C. These aliquots are designated as generation-1 (G-1).

One aliquot of G-1 was inoculated into ~10 mL of MHBII and grown aerobically for several hours at 35-37° C. up to OD₆₀₀~0.8-1. The culture was then cooled to room temperature and a ~10-fold dilution was prepared using a portion of the fresh culture, so that the new diluted suspension reaches an OD~0.1. Both the OD=0.8-1 culture and the OD~0.1 suspension were added with Tw80 up to final concentration of 0.1%, homogenized by vortexing and stored at 4° C. 100 μL from the OD~0.1 suspension was 10-fold serial diluted in dilution buffer and inoculated on to TSB plates as spreads. The TSB plates were incubated at 30-37° C. overnight. The colonies formed on the TSB plates were enumerated to estimate the concentration of viable bacteria in the OD~0.1 suspension in terms of colony forming units (CFU). This OD~0.1 suspension was used to spike buffers and media for experiments concerning microbial capture, growth and biofilm formation.

### Example 3: Charge of Particles and Microbes

The charge of particles and microbes were determined by Diffraction Light Scattering (DLS) using Zetasizer Nano (Malvern Panalytical). Small aliquots of the NT particles were dispersed in water, and the suspensions were used conducting the DLS.

5 ml of microbial suspension (OD~1) was precipitated by centrifugation and washed in 5% glucose with 0.1% Tw80 solution two times and then suspended in 1 mL of 5% glucose with 0.1% Tw80 solution. This process washes off the salts that may interfere with the zeta potential measurement studies and gets the microbes suspended in physiologically relevant osmotic conditions while mitigating their aggregation. Small aliquots of the microbial suspensions were dispersed in water, and the suspensions were used conducting the DLS.

The zeta potentials of the particles were, as expected, found to be +35 to +50 mV, demonstrating that the particle surfaces carry high electropositive charge. On the other hand, the zeta potentials of the microbes were −35 to −38 mV, demonstrating that the microbial cell surfaces carry high electronegative charge. The results indicate that NT particles are highly poised to attract and bind microbial cells when brought in proximity inside a liquid sample.

TABLE 2

| Sample | Zeta Potential (mV) |
|---|---|
| NT-B | +36.41 |
| NT-C | +49.48 |
| E. coli 25922 | −35.05 |
| C. albicans 10231 | −37.89 |

### Example 4: Capture of Microbes and Culturing in Presence of Magnetic Hydrogel Particles

HBBS was spiked with Klebsiella pneumoniae ATCC 13884 (Klebsiella p. 13884) up to a final concentration of 10′ CFU/mL and distributed as 5 mL aliquots. The aliquots were added with 75 μL of NT-B and NT-C separately in duplicate and incubated at room temperature for ~30 minutes. Then the particles were magnetically separated, and supernatants were collected in fresh tubes. The particles were added with 300 μL Mod. BHI V-1. 10-fold serial dilutions of the bacteria were prepared in Mod. BHI V-1 and HBBS separately. Then the whole volumes of the NT samples were pipetted into separate wells of a 96 well black pp microplate. 150 μl from the supernatants were added to separate wells followed by 150 μL of 2×NBB. For standard curves, in separate wells of a column NT particles diluted in Mod. BHI V-1 were added, so that the mass of the particles in each of these wells was equal to the mass of the particles in the sample wells. In a separate column 150 μL of 2×NBB was added. To the wells for the standard curves, 150 μL of the bacterial dilutions were then added—in BHI V-1 for in presence of NT and in HBBS for in absence of NT. 15 μL of aqueous resazurin solution (800 μg/mL) was added to all these wells. The empty wells were filled with 300 μL of water. The plate with covered with clear lid and the gap between the plate and the lid was sealed with parafilm. Then the whole plate was incubated at 35° C., overnight, with continuous shaking and intermittent fluorescence read at 540 nm/590 nm.

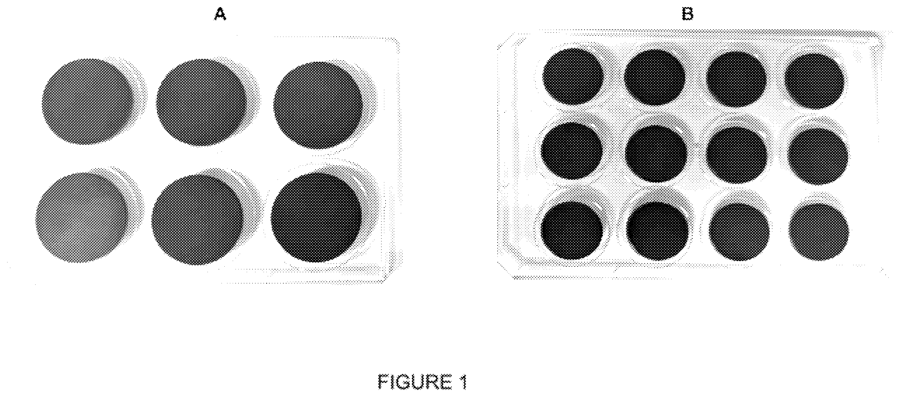

FIG. 1 shows the results obtained from this experiment. On the left side of the figure are correlations between the threshold time and starting bacterial populations. The threshold times are the times required for the samples to reach ~15% of the maximum fluorescence reached during the overnight incubation period. The correlations show that threshold times are linearly correlated to the log₁₀(initial CFUs) in the corresponding wells. These correlations afforded us to estimate the initial bacterial population in the wells. The results show that the supernatants were almost depleted of cells after incubation with the NT particles. However, the number of viable cells present in association with NT-B immediately post incubation was ~1% of the spike, while NT-C showed ~4.5× survival of cells w.r.t the spike. This indicates that NT-B may be detrimental to the viability of cells, while NT-C may exert high cell proliferation effect on the captured cells.

### Example 5: Capture and Growth of Biofilm of Fluorescent Protein Producing Cells to Determine Population of Microbes in the Harvested Biofilms

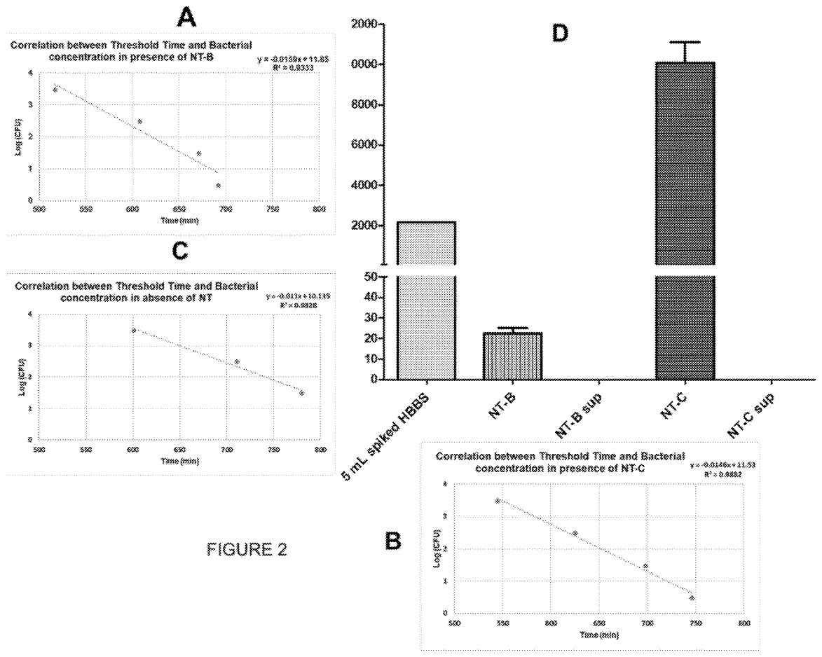

E. coli 25922GFP was grown in MHBII supplemented with 0.4 mg/mL Amp and 1 mM IPTG overnight at 35° C. up to an OD~1.5. Diluted sample from this culture (OD~1.4) was prepared and the CFU concentration of the suspension was determined according to the methods described in Example 4. The OD~1.4 suspension was spiked in HBBS up to a final concentration of 10⁵ CFU/mL and distributed as 5 mL aliquots in 5 mL tubes. 5 mL of aliquots of un-spiked HBBS were also prepared in 5 mL tubes. The aliquots were added with 250 μL of NT-B and NT-C separately in duplicate and incubated at room temperature for ~30 minutes. Then the particles were magnetically separated, and the supernatants were discarded. The particles were suspended in 2 mL LB broth and transferred to the wells of a 12 well microplate (FIG. 3)—column 1: NT-B, column 2: NT-C, where rows A and B are bacteria positive samples, while row C was bacteria negative samples. Fresh LB broth was spiked to 2.5×10⁵ CFU/mL and 2 mLs of this spiked broth were transferred to each of two wells (3A and 3B) of the microplate. Sterile LB was pipetted to well 3C. The other wells were filled with water. The plate was incubated overnight at 37° C. without shaking. The plate was then place on a 12 well magnetic device (FIG. 2) and the supernatants were carefully pipetted out and discarded. The wells were carefully washed with HBBS two times and then added with 2 mL of fresh HBBS. A photograph (FIG. 3 left) was taken by illuminating the plate with near UV light and placing an orange filter between the plate and the camera lens. Then the contents of the wells were resuspended and homogenized by repeated pipetting up and down and then collected in separate 2 mL tubes. E. coli 25922GFP suspension OD~1.5 was centrifuged and resuspended in HBBS. Then 2-fold serial dilutions were prepared from this sample.

To a 96 well black pp microplate, 100 μL of the stored samples from the 2 mL were added. For standard curves, NT particles were suspended in HBBS and added to specific columns of the plate, so that the mass of the particles in these wells matched with the mass of the particles in the sample wells. The wells were then added with suspensions of serially diluted bacteria in HBBS—the total volumes being 100 uL In the wells of a separate column, 50 μL of fresh HBBS was added followed by 50 uL suspensions of serially diluted bacteria in HBBS. The wells for the standard curve were then added with serial dilutions of the bacteria. Fluorescence was read at 490 nm/515 nm for the eGFP protein.

The picture shows green glowing thin biofilm suspended in the wells (3A and 3B) without NT, while the bacteria negative wells do not show any floating biofilm. The floating nature of the biofilm indicates that the biofilm may have matured and cannot firmly adhere to surfaces. The fluorescence read results indicate similar degrees of presence of bacterial cells among all the bacteria positive samples—NT particle samples have similar amounts of bacterial population as non-NT particle samples. By combining this two information, it may be inferred that while without NT presence, the bacteria are embedded in the biofilm loosely bound to plastic surfaces of the wells, in presence of NTs, bacteria and possibly their biofilm are firmly attached to the surfaces of the particles.

### Example 6: Capture of Microbe by Magnetic Hydrogel Particles and Growth of Biofilm

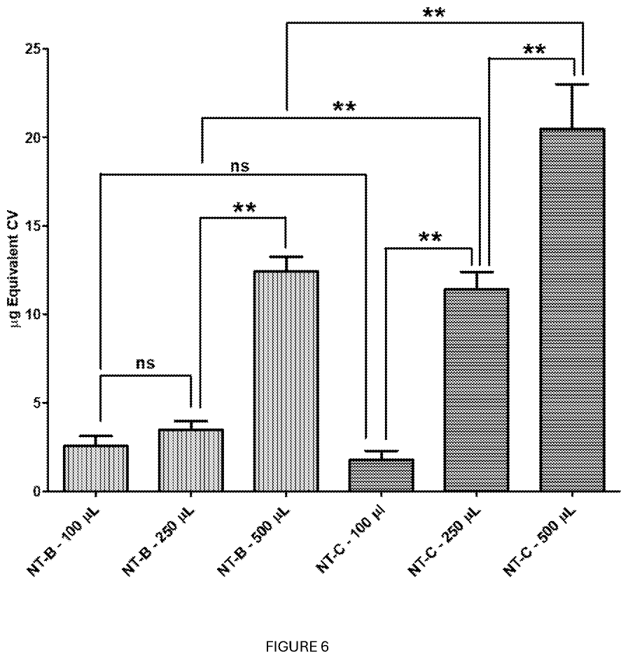

In one attempt, HBBS was spiked with Klebsiella p. 13884 up to a final concentration of 10¹ CFU/mL and distributed as 5 mL aliquots in each of 8 tubes. 5 mL of aliquots of un-spiked HBBS were also prepared in each of four 5 mL tubes. The aliquots were added with 250 μL NT-C separately and incubated at room temperature for ~30 minutes. Then the particles were magnetically separated, and supernatants were discarded. Then the particles were resuspended separately in 2 mL of four different broths—LB, BHI, Mod. BHI V-2 and Mod. BHI V-4 and then pipetted into separate wells of a 12 well plate. The plate was incubated overnight at 37° C. for ~24 hours. Then the plate was placed on appropriate magnetic device (Figure-1) and the supernatants were pipetted out and discarded. The supernatants from bacteria positive samples were highly turbid indicating growth of bacteria, while the supernatants from the bacteria negative samples were transparent indicating sterility of the samples. The particles were then washed two times with HBBS, and the samples were stained using 0.01% aqueous CV solution for 30 minutes. Then the samples were washed several times with HBBS to get rid of extra stains. The samples were briefly dried at room temperature, before addition of 300% acetic acid. The CV-stained biofilms were dissolved into the dil. AcA by incubation at room temperature and shaking at 100 rpm. The particles were separated under magnetism, and the supernatants were collected in separate tubes. 75 μL of each of these sample solutions were pipetted in separate wells of a clear 96 well pp microplate and absorbances were measured at 586 nm. The results (FIG. 6) show production of biofilm in all the samples spiked with bacteria with respect to samples not spiked with bacteria.

In another attempt, HBBS was spiked with Klebsiella p. 13884 up to a final concentration of 10⁵ CFU/mL and distributed as 5 mL aliquots in each of 16 tubes. 5 mL of aliquots of un-spiked HBBS were also prepared in each of eight 5 mL tubes. 500 μL of NT-B was added to 8 of the aliquots containing spikes while the other 8 spiked aliquots were added with 500 μL of NT-C. Four non-spiked aliquots were added with NT-B while the other four with NT-C. All the tubes containing the NT samples were incubated by rolling at room temperature for 30 minutes, after which the particles were separated by magnetism and the supernatants were discarded. The tubes were then segregated according to the NT-content—NT-B or NT-C, and for within each NT-set, two subsets were created. Each subset had 4 tubes which had bacteria spikes and two tubes, which did not have bacteria spikes. 2 mL LB broth was added to one subset, while 2 mL mod. BHI V-4 was added to the other subset. All the contents of the tubes were pipetted to separate wells of 12 well microplates. The microplates were incubated at 37° C. overnight for ~24 hours. The next day, the microplates were placed on appropriate magnetic device (Figure-1) and the supernatants were pipetted out and discarded. The supernatants from bacteria positive samples were highly turbid indicating growth of bacteria, while the supernatants from the bacteria negative samples were transparent indicating sterility of the samples. The particles were then washed two times with HBBS, and the samples were stained using 0.05% aqueous CV solution for 30 minutes. Then the samples were washed several times with HBBS to get rid of extra stains. The samples were briefly dried at room temperature, before addition of 30% acetic acid. The CV-stained biofilms were dissolved into the dil. AcA by incubation at room temperature and shaking at 100 rpm. The particles were separated under magnetism, and the supernatants were collected in separate tubes. 75 μL of each of these sample solutions were pipetted in separate wells of a clear 96 well pp microplate and absorbances were measured at 586 nm. The results show production of biofilm in all the samples spiked with bacteria with respect to samples not spiked with bacteria. When the NT and the growth media were compared (FIG. 6) significant differences were observed. For each growth media, NT-C demonstrated higher biofilm harvest that NT-B. The biofilm harvest by NT-B were similar for both LB and mod. BHI V-2, while the biofilm harvest by NT-C using mod. BHI V-2 was significantly higher than using LB broth. The result indicated that while both NT-B and NT-C can harvest biofilm, NT-C demonstrated higher capability of such harvest. On the other hand, the growth media can affect the degrees of biofilm formation by a microbe.

### Example 7: Biofilm Harvest by Different Volumes of Magnetic Hydrogel Particles

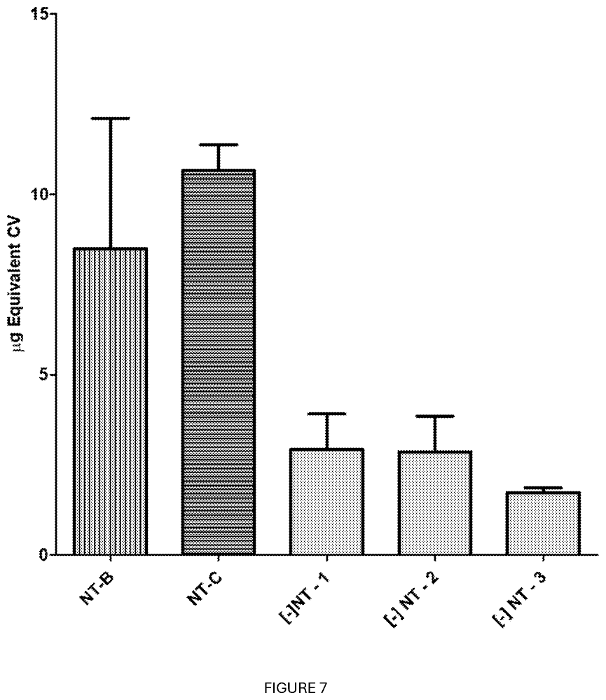

HBBS was spiked with Klebsiella p. 13884 up to a final concentration of 10⁵ CFU/mL and distributed as 5 mL aliquots and added with 100 μL, 250 μL and 500 μL of NT-B and NT-C separately in triplicate along. Unspiked 5 mL HBBS aliquots were also added with same volumes of NT particles. The samples were incubated by rolling at room temperature for 30 minutes, after which the particles were separated by magnetism and the supernatants were discarded. Then the particles were resuspended separately in 2 mL of BHI broth and pipetted into separate wells of 12 well microplates. The plates were incubated at 37° C. overnight following which the particles were separated under magnetism and the supernatants were discarded. The particles were then washed two times with HBBS, and the samples were stained using 0.05% aqueous CV solution for 30 minutes. Then the samples were washed several times with HBBS to get rid of extra stains. The samples were briefly dried at room temperature, before addition of 30% acetic acid. The results (FIG. 7) show variable harvest of biofilm by different volumes of NT particles. 100 μL and 250 μL of NT-B demonstrated similar degrees of biofilm harvest while 500 μL of NT-B showed significantly higher amounts of biofilm harvest. Among the amounts of biofilm harvest by NT-C, 100 μL showed the least amounts, while 250 μL showed intermediate amounts and 500 μL showed the highest amounts. When the biofilm harvest by same volumes of NT-B and NT-C are compared, there is no significant difference in biofilm amounts between their 100 μL volumes. On the other hand, for other two volumes, biofilm harvests by NT-C were higher than that by NT-B.

### Example 8: Harvest of Biofilm by Magnetic Hydrogel Particles and Subsequent Subculturing

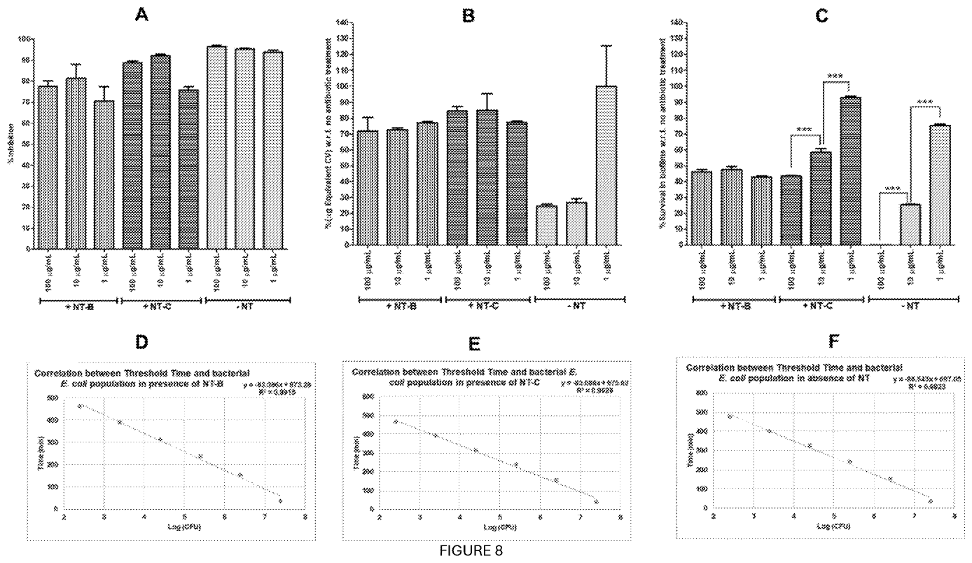

HBBS was spiked with Klebsiella p. 13884 up to a final concentration of 10⁵ CFU/mL and distributed as 5 mL aliquots and added with 300 μL of NT-B and NT-C separately in 4 replicates. Two replicates of non-spiked HBBS were also added with 300 μL of each of NT-B and NT-C. The samples were incubated by rolling at room temperature for 30 minutes, after which the particles were separated by magnetism and the supernatants were collected in separate tubes. The NT samples were then added with 3 mL of BHI broth and transferred to 12 well microplates. Another sample of HBBS was spiked with 2.5×10⁵ CFU/mL and 2 mL of this suspension was pipetted to each of two wells of each microplate. The supernatants, along with a portion of unused spiked HBBS and another HBBS sample spiked with 5×10⁵ CFI/mL were stored at 4° C. The plates were incubated at 37° C. overnight following which the particles were separated under magnetism and the supernatants were collected in separate tubes and stored at 4° C. The supernatants above the biofilms produced in the (−) NT sample wells were carefully pipetted out and stored at 4° C. All the supernatants from samples with bacteria spikes were highly turbid—indicating growth of bacteria, while the supernatants from samples without bacteria spikes were transparent—indicating sterility of the samples. Two replicates from each of the NT samples and the biofilms produced without NT were resuspended in fresh BHI and transferred to sterile culture tubes, followed by addition of more fresh BHI broth up to 10 mL; these samples were incubated at 35° C. overnight with 200 rpm shaking. On the other hand, the remaining replicates from each of the NT samples and the biofilms produced without NT were stained by CV using common staining, destaining and solubilizing procedures followed so far. The next day, the magnetic particles were separated by magnetism, and the supernatants (designated as Subculture-1) were stored at 4° C. The subcultures from (−) NT samples were also stored at 4° C. The magnetic particles were added with fresh BHI broth and incubated at 35° C. overnight with 200 rpm shaking. By repeated separation of particles and subculturing, Subculture-2 and Subculture-3 were collected. One replicate from each of NT-C samples was selected and 500 μL of that sample was used for DNA extraction. 500 μL of stored unused spiked samples—10⁵ CFU/mL and 5×10⁵ CFU/mL and non-spiked were also subjected for DNA extraction. Nucleic acids were extracted using the Nanotrap® PRIME Extraction Kit and quantified using singleplex qPCR assays targeting Klebsiella pneumoniae. The assay was selected from a manufacturer-validated TaqMan® Microbe Detection Assay (ID Ba04932083_s1) provided by Thermo Fisher Scientific and implemented according to the manufacturer's specifications. FIG. 8 shows the amounts of biofilm harvest by different samples. It was evident that both NT-B and NT-C harvest biofilms. Even though the means of the amounts of biofilm harvest using NT particles were higher than that without using NT particles, due to small number of replicates (n=2), statistical difference could not be estimated. The supernatants of subculture-1 with bacteria spike had ODs in the range of 1.3-1.5 while the ODs of subcultures 2 and 3 were ~2. The high turbidity indicates that repeated subculturing was possible from the captured cells in presence of NT particles. Table 3 shows the Cₜ values from qPCR of tested samples for Klebsiella gene. The spiked matrix (1×10⁵ CFU/mL) provided a Cₜ value of 28.2 while that with 5×10⁵ CFU/mL provided a Cₜ value of 24.8. The amount of bacteria remaining in the supernatant collected after capture by NT-C was very low—a Cₜ value of 32.0. This result matches closely with Example 4, which demonstrated near absence of viable cells in the supernatants after incubation with NT particles. By combining that result with the present one, it can be inferred that NT particles capture most of the bacterial population from the liquid sample. Both the supernatant and the biofilm harvested by NT-C had significant population of bacteria—with the concentration of bacteria higher in the biofilm (Cₜ=18.5) than in the supernatant (Cₜ=21.0). The subcultures collected over NT-C incubated with spiked matrix demonstrated presence very high bacterial population (with Cₜ=18-20). Interestingly, the corresponding NT-C samples incubated with non-spiked matrix also displayed presence of minute amounts of Klebsiella gene (Cₜ=29-32). This minute amount of Klebsiella gene must be coming from random contamination of non-viable bacteria or bacterial nucleic acids, either present in the original NT stock or in the broth or in both. This is because as part of synthesis process, the NT particles were appropriately washed to make them sterile. Moreover, 500 μL of NT particles were incubated with fresh MHBII at 37° C. for two days—no change in turbidity of the broth was observed, indicating sterility of the NT particle stocks.

TABLE 3

Ct values

| Sample Name | Average | Std Dev |
|---|---|---|
| spiked matrix (1 × 10\hat{ }5 | 28.2 | 0.11 |
| CFU/mL) | | |
| supernatant after capture | 32.0 | 0.01 |
| Elution Buffer | 38.2 | 1.62 |
| spiked matrix (5 × 10\hat{ }5 | 24.8 | 0.03 |
| CFU/mL) | | |
| +NT (+)biofilm | 18.5 | 0.00 |
| Sup from +NT (+)biofilm | 21.0 | 0.03 |
| Culture 1 (sup of biofilm) | 19.3 | 0.12 |
| Culture 2 (sup of biofilm) | 18.1 | 0.11 |
| Culture 3 (sup of biofilm) | 18.4 | 0.11 |
| +NT (-)bacteria | 31.6 | 0.01 |
| Sup from +NT (−)bacteria | 29.4 | 0.03 |
| Culture 1 {+NT (−)bacteria} | 29.8 | 0.04 |
| Culture 2 {+NT (−)bacteria} | 29.6 | 0.02 |
| Culture 3 {+NT (−)bacteria} | 29.6 | 0.01 |

### Example 9: Antimicrobial Susceptibility Testing on the Formed Biofilm Harvested by Magnetic Hydrogel Particles

HBBS was spiked with E. coli 25922 up to a final concentration of 5×10⁵ CFU/mL and distributed as 5 mL aliquots and added with 250 μL of NT-B and NT-C separately in 12 replicates for each NT. The samples were incubated by rolling at room temperature for 30 minutes, after which the particles were separated by magnetism and the supernatants were discarded. The NT samples were then added with 3 mL of LB broth and transferred to 12 well microplates. A fresh volume of LB broth was spiked with 5×10⁵ CFU/mL; 3 mL of this suspension were transferred to each of the wells of a 12 well microplate. All the plates were incubated at 37° C. for 24 hours. The supernatants over the biofilms were carefully pipetted out and discarded. The biofilms were carefully washed 2 times with fresh HBBS. Then the wells were added with 3 mLs of 100 μg/mL, 10 μg/mL, 1 μg/mL and 0 μg/mL of levofloxacin (LVFX) in triplicate and incubated at 35° C. for 24 hours. The supernatants from the wells were collected and stored in tubes. The biofilms were carefully washed two times with fresh HBBS. Then fresh MHBII was added to one of the 3 replicates, suspended by pipetting and then transferred to a tube. The other two replicates were stained by CV (0.01%) using common staining, destaining and solubilizing procedures followed so far. 200 μL from the saved biofilm suspensions were pipetted into separate wells a 96 well black pp microplate. The bacterial survival in the biofilms was estimated by resazurin assay outlined in Example 6. 100 μL from the supernatants over biofilms collected after incubation with varying concentrations of LVFX were pipetted to separate wells of a clear 96 well microplate and the absorbance was read at 600 nm. Based on the absorbances, % inhibition of bacterial growth by different concentrations of LVFX were estimated with respect to samples treated with no antibiotic.

The results (FIG. 8A) show that LVFX had inhibitory effect on generation of planktonic cells in all the samples across the three concentrations tested. In biofilms produced without NT particles, the degrees of inhibition were >90%, whereas they vary between 70-90% for biofilms produced on NT-B samples. In case of biofilms produced on NT-C, 100 μg/mL and 10 μg/mL of LVFX inhibited generation of microbial cells by >90%, while for 1 μg/mL the degrees of inhibition were reduced to ~70%.

The degrees of biofilm eradication (FIG. 8B) by LVFX varied significantly dependent on whether the biofilms were present in association of NT or not. The results showed that NT particles preserved biofilm with 70-90% efficacies against antibiotic treatment. On the other hand, bare biofilms were significantly eradicated with 100 μg/mL and 10 μg/mL of LVFX treatment, while 1 μg/mL treatment had highly variable degrees of biofilm eradication among the replicates.

The threshold times were linearly correlated with initial bacterial concentrations (FIG. 8D, E, F). These correlations afforded us to estimate surviving bacterial population the biofilm samples post antibiotic treatment. The degrees of bacterial survival in the biofilm post antibiotic treatment were highly varied among the samples (FIG. 8C). The degrees of survival were reduced to 40-50% in the biofilms associated with NT-B, while increasing concentrations of antibiotic resulted in reduced bacterial survival in the biofilms associated with NT-C—a similar trend that was observed in case of biofilms produced in absence of NT particles. This indicates that antimicrobial susceptibility test (AST) results involving biofilms harvested by NT-C can replicate the trend expected in AST conducted on regular biofilms produced without magnetic hydrogel particles.

### Example 10: Capture of Yeast Cells and Harvest of Biofilms by Magnetic Hydrogel Particles

Candida albicans 10231 was grown in TSB at 35° C. up to OD=1.4. 20 μL of this culture was spiked into 40 mL of fresh HBBS as 5 mL aliquots in each of 6 tubes. 5 mL of aliquots of un-spiked HBBS were also prepared in each of two 5 mL tubes. The aliquots were added with 250 μL NT-B and NT-C separately and incubated at room temperature for ~30 minutes. Then the particles were magnetically separated, and supernatants were discarded. Then the particles resuspended separately in 3 mL TSB supplemented with 5% glucose and transferred to the wells a 12 well microplate. 10 mL of fresh (TSB+5% glucose) was spiked with 25 μL of the culture and 3 mL of this spiked sample was pipetted to two separate wells, with another separate well was filled with 3 mL of non-spiked (TSB+5% glucose). The plate was incubated at 35° C. for 24 hours. The supernatants were carefully pipetted out and discarded. The biofilms were washed two times with fresh HBBS. Then the biofilm contents of the wells were stained by CV (0.01%) using common staining, destaining and solubilizing procedures followed so far. 75 μL of the samples were pipetted in to separate wells of a 96 well clear pp microplate and absorbance read at 585 nm.

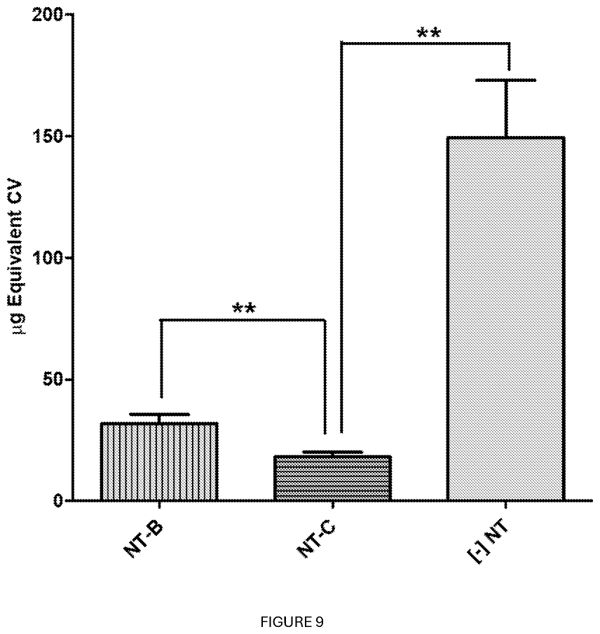

The results (FIG. 9) show that both NT-B and NT-C harvest yeast biofilm in similar degrees, while the biofilm harvested without NT was significantly high.

Having illustrated the present invention, it should be understood that various adjustments and versions might be implemented without venturing away from the essence of the present invention. Further, it should be understood that the present invention is not solely limited to the invention as described in the embodiments above, but further comprises any and all embodiments within the scope of this application.

The foregoing descriptions of specific embodiments of the present invention have been presented for purposes of illustration and description. They are not intended to be exhaustive or to limit the present invention to the precise forms disclosed, and obviously many modifications and variations are possible in light of the above teaching. The exemplary embodiment was chosen and described in order to best explain the principles of the present invention and its practical application, to thereby enable others skilled in the art to best utilize the present invention and various embodiments with various modifications as are suited to the particular use contemplated.

We claim:

1. A type of magnetic hydrogel particle wherein the hydrogel material is coated on magnetic core, which is composed of magnetic nanoparticle infused polymeric microparticle, and the hydrogel material is connected to the polymeric material via covalent chemical bonds and wherein,
a. the hydrogel material is either doped with any or two or multiple of the following cationic groups—substituted or non-substituted amine groups (—NH₂, —NH—R—, —R¹—(N—)—R²—, (—N+)—(R¹—)(R²—)(R³—)) or imine groups (>C═NH, >C═N—R), guanidinium group [(R¹R²N)(R³R⁴N)C═NR⁵], pyridinium group (C₅H₅N⁺R), sulfonium group (—S⁺R¹R²R³), carbenium and oxonium groups,
b. or the hydrogel material is doped with any or two or multiple of the following anionic groups—carboxyl (—COOH) group, sulfonate and sulfate (RSO₃⁻ and ROSO₃⁻ respectively) groups, phosphate (ROPO₃²⁻) group, or alkoxide (RO⁻) group,
c. or the hydrogel material is doped with one or two or multiple zwitterionic moieties, which contain one or multiple of the mentioned cationic groups and one or multiple of the mentioned anionic groups,
where “R” s represent hydrogen, or alkyl, alkenyl, or aromatic group, which may be bonded or non-bonded to other organic moieties.

2. A method of creating magnetic hydrogel particle wherein the hydrogel material is coated on magnetic core composed of magnetic nanoparticle infused polymeric microparticle, and the hydrogel material is connected to the polymeric material via covalent chemical bond and wherein,
a. The hydrogel material is either doped with any or two or multiple of the following cationic groups—substituted or non-substituted amine groups (—NH₂, —NH—R—, —R₁—(N—)—R²—, (—N+)—(R₁—)(R²—)(R³—)) or imine groups (>C═NH, >C═N—R), guanidinium group [(R¹R²N)(R³R⁴N)C═NR⁵], pyridinium group (C₅H₅N⁺R), sulfonium group (—S⁺R¹R²R³), carbenium and oxonium groups;
b. or the hydrogel material is doped with any or two or multiple of the following anionic groups—carboxyl (—COOH) group, sulfonate and sulfate (RSO₃⁻ and ROSO₃⁻ respectively) groups, phosphate (ROPO₃²⁻) group, or alkoxide (RO⁻) group; and
c. or the hydrogel material is doped with one or two or multiple zwitterionic moieties, which contain one or multiple of the mentioned cationic groups and one or multiple of the mentioned anionic groups,
where “R”s represent hydrogen, or alkyl, alkenyl, or aromatic group, which may be bonded or non-bonded to other organic moieties.

3. An approach for binding cellular target in a liquid medium by
a. providing a liquid medium including the cellular targets
b. providing hydrogel coated magnetic particles of claim 1, capable of directly binding the cellular target in the liquid medium via non-covalent interactions
c. forming a complex of the magnetic hydrogel particles of claim 1 and the cellular target by combining the magnetic hydrogel particles and the liquid medium, the cellular target material being directly adhered to the hydrogel material of the particles
wherein, the cellular target may be one or multiple of the following possibilities:
i. nucleic acid,
ii. microbial cell,
iii. animal cell,
iv. plant cell,
v. protein or a peptide,
vi. carbohydrate,
vii. extracellular vesicles,
viii. component of biofilm,
ix. metabolite,
x. lipid nanoparticles,
xi. mitochondria,
xii. cell nucleus,
xiii. chloroplast, and
ixx. Golgi bodies.

4. An approach for binding cellular target in claim 3 and separating the complex from the liquid medium by either application of an external magnetic field or by precipitation under centrifugal force or gravitational force.

5. An approach for binding cellular target and separating the complex of magnetic hydrogel particles and a cellular target or multiple cellular targets in claim 3 and separating a cellular target or multiple cellular targets from the complex whereby the isolated cellular target or multiple cellular targets are obtained.

6. An approach for binding animal cells in a liquid medium according to claim 3, wherein the animal cells bound according to the approach consists of cancerous type.

7. An approach for isolating animal cells in complexation with magnetic hydrogel particles according to claim 4, wherein, the animal cells bound according to the approach consists of cancerous type.

8. An approach for isolating animal cells from a liquid medium according to claim 5, wherein the animal cells bound according to the approach consists of cancerous type.

9. An approach for isolating microbial cells or animal cells or plant cells in complexation with magnetic hydrogel particles from a liquid medium according to claim 4 and bringing the complex in contact with a fresh volume of same or another liquid for the purpose of preserving or proliferating the isolated cells.

10. An approach for preserving or proliferating animal cells according to claim 9, wherein the animal cells are of cancerous type.

11. An approach for isolating cellular target in complexation with magnetic hydrogel particles according to claim 4 and bringing the complex in contact with another liquid buffer or medium for preservation of the components of the complex or for proliferation, identification, analysis or manipulation or a combination of these purposes for the nucleic acid.

12. An approach for isolating microbial cells in association with magnetic hydrogel particles and bringing them in contact with suitable liquid medium according to claim 4 for the purpose of proliferation and production of biofilm.

13. An approach for production of biofilm in presence of magnetic hydrogel particles according to claim 12 for the purpose of identification, characterization, analysis, manipulation or a combination of these purposes of the components of the biofilm.

14. An approach for production of biofilm in presence of magnetic hydrogel particles according to claim 12 and bring the biofilm in contact with other chemical substances in presence or absence of the magnetic particles to study the effect of those substances, which may but not limited to drugs or antimicrobial agents, on the components of biofilm.

15. An approach for isolating microbial cells from a liquid medium and bringing the cells in contact with suitable liquid medium according to claim 5 for the purpose of proliferation and production of biofilm.

16. An approach for production of biofilm in presence of magnetic hydrogel particles according to claim 12 for the purpose of identification, characterization, analysis, manipulation or a combination of these purposes of the components of the biofilm.

17. An approach for production of biofilm in presence of magnetic hydrogel particles according to claim 12 and bring the biofilm in contact with other chemical substances, which may but not limited to drugs or antimicrobial agents, in presence or absence of the magnetic particles to study the effect of those substances on the components of biofilm.

18. An approach for binding cellular targets according to claim 3 and bringing two or multiple cellular targets in close proximity of each other, wherein
a. one cellular target can be nucleic acid;
b. one cellular target can be microbial cell;
c. one cellular target can be animal cell;
d. one cellular target can be plant cell;
e. one cellular target can be a protein or a peptide;
f. one cellular target can be carbohydrate;
g. one cellular target can be extracellular vesicles;
h. one cellular target can be metabolite;
i. one cellular target can be lipid nanoparticles;
j. one cellular target can be nucleus;
k. one cellular target can be mitochondria;
l. one cellular target can be chloroplast; and
m. one cellular target can be Golgi bodies.

19. An approach for binding cellular targets according to claim 3, wherein one type of cellular target is microbial cell or animal cell or plant cell or extracellular vesicle or lipid nanoparticle, and wherein the magnetic hydrogel particle eventually gets internalized into the cell or the extracellular vesicle or the lipid nanoparticle.

20. An approach for binding cellular targets and bringing two or multiple cellular targets in close proximity of each other according to claim 18, and eventually one or multiple cellular targets out of the following possibilities:
a. nucleic acid,
b. protein or peptide,
c. carbohydrate,
d. lipid,
e. metabolite,
f. extracellular vesicle,
g. virus,
h. lipid nanoparticles,
i. nucleus,
j. chloroplast, and
k. mitochondria or a drug in complexation or not in complexation with the magnetic hydrogel particle get internalized into extracellular vesicle, lipid nanoparticle, nucleus, mitochondria, chloroplast, microbial, animal or plant cells.
